(19) United States
(12) Patent Application Publication
(10) Pub. No.: US 2026/0144841 A1
(43) Pub. Date: May 28, 2026

(54) USE OF ARECA CATECHU PRODUCT AS SOLE ACTIVE INGREDIENT IN MANUFACTURING BIOLOGICAL FEED AND DINKING PREPARATION FOR PREVENTING AND CONTROLLING AFRICAN SWINE FEVER

(71) Applicant: CHANGSHA LVYE BIOTECHNOLOGY CO. LTD., Changsha (CN)

(72) Inventors: Lixin WEN, Changsha (CN); Xiaohong XIE, Changsha (CN); Xinrong DONG, Changsha (CN); Wei DONG, Changsha (CN); Rongfang LI, Changsha (CN); Linyu ZHANG, Changsha (CN); Xianglin WANG, Changsha (CN); Jinhui MAI, Changsha (CN); Xin LI, Changsha (CN)

(21) Appl. No.: 18/962,172

(22) Filed: Nov. 27, 2024

(51) Int. Cl. A61K 36/889; A01N 65/40; A01P 1/00; A61P 31/20

(52) U.S. Cl. CPC A61K 36/889; A01N 65/40; A01P 1/00; A61P 31/20

(57) ABSTRACT

The present disclosure belongs to the technical field of biological feeds, and in particular relates to use of an Areca catechu product as a sole active ingredient in manufacturing a biological feed and a dinking preparation for preventing and controlling African swine fever. In the present disclosure, the Areca catechu product as the sole active ingredient is mixed with an African swine fever virus (ASFV) suspension, and then inoculated in cells. Evaluation by in vitro cell experiments demonstrates that the Areca catechu product as the sole active ingredient has a good killing effect on ASFV, where 100% of ASFV with a virus titer of 5×10³ HAD₅₀/mL and can be killed within 30 minutes, and 100% of ASFV with a virus titer of 5×10⁴ HAD₅₀/mL can be killed within 2 hours.

### TECHNICAL FIELD

The present disclosure belongs to the technical field of biological feed, and in particular relates to use of an Areca catechu product as the only active ingredient in manufacturing biological feed and/or dinking preparation for preventing and controlling African swine fever.

### BACKGROUND

African Swine Fever (ASF) is an acute, febrile and highly contagious zoonotic disease of pigs caused by African Swine Fever Virus (ASFV). The morbidity and mortality of pigs infected with ASFV can be as high as 100%. At present, several ASFV variants with low mortality have emerged, exhibiting covert clinical manifestations while still posing a risk of horizontal transmission. They result in persistent infection, chronic course of disease and even death, which further increase the complexity and difficulty in the prevention and control of the diseases caused by the ASFV variants. For the prevention and control strategy of viral diseases, vaccination has always been the preferred strategy, but so far, there is no commercial vaccine to prevent African swine fever.

The most important transmission route of ASFV is oral transmission, with contaminated pig feed and water source being the main transmission media. To block the feed-borne transmission of ASFV, an effective biosafety method currently in use is high-temperature pelleting at 85° C. for 3 minutes to kill viruses in feed. This method requires significant investment, notably decreases the production efficiency, increases feed cost per ton by around 80 yuan, and can damage the heat-sensitive nutrients in the feed, such as multivitamins. ASFV has a lower infection threshold and a higher risk when transmitted through water compared to its spread though feed, so contaminated water sources in pig farms are more likely to cause infection and morbidity in pig herds. To block the drinking water-borne spread of ASFV, the commonly used method at present is to add disinfectants (such as potassium persulfate or sodium hypochlorite) or acidifiers to drinking water. At present, there is no research to confirm that acidifiers have a complete killing effect on ASFV, and the disinfectant needs to reach a certain concentration to kill ASFV. Maintaining chemical disinfectants at a high concentration in drinking water for a long time will inevitably damage the oral cavity and intestinal mucosa and disturb the intestinal flora in pig herds, making pigs more susceptible to infection and disease.

Areca catechu is an evergreen tree of Areca genus in the family of Arecaceae, and it mainly grows in the tropical and subtropical regions in southern China and other countries in South Asia and Southeast Asia. It is one of the traditional authentic medicinal materials and important medicinal plants in China. The seeds, pericarp, flowers, etc. of Areca catechu all can be used as medicine. The raw fruits of Areca catechu contain a variety of physiologically active ingredients, mainly including alkaloids, polyphenols, polysaccharides, fatty acids, etc. Modern studies have shown that Areca catechu has a wide range of pharmacological effects, exhibiting a variety of biological activities such as promoting digestion, antioxidant activity, anti-inflammatory activity, anti-parasitic activity and bacteriostatic activity impacting the digestive system, nervous system, cardiovascular system and endocrine system of human body. However, there's no research identifying the ASFV killing effect of Areca catechu and its extract.

### SUMMARY

In order to solve the deficiencies in the conventional technology, an objective of the present disclosure is to provide the use of an Areca catechu product as a sole active ingredient in manufacturing a biological feed and/or a dinking preparation for preventing and controlling ASF, where the Areca catechu product is added to a feed and/or a water source to kill ASFV in the feed and drinking water, thereby cutting off the feed-borne transmission and drinking water-borne transmission of ASF.

The present disclosure provides the use of an Areca catechu product as a sole active ingredient in manufacturing a biological feed and/or a dinking preparation for preventing and controlling ASF.

In some embodiments, the Areca catechu product includes Areca catechu extract and/or Areca catechu ultrafine powder.

In some embodiments, a content of polyphenol in the Areca catechu extract is 2% to 30%; and
the Areca catechu ultrafine powder is an undersize of a sieve with meshes of ≥80.

In some embodiments, a method for preparing the Areca catechu extract includes the steps of:
mixing an Areca catechu raw material with an extraction solvent, performing extraction, and collecting liquid extract of Areca catechu; and
subjecting the liquid extract of Areca catechu to drying to obtain the Areca catechu extract.

In some embodiments, a mass-to-volume ratio of the Areca catechu raw material to the extraction solvent is 1 g:(8 to 35) mL;
the drying is performed at a temperature of 65 to 75° C. for 72 to 96 h.

In some embodiments, the extraction solvent is any one of water, methanol, and an aqueous hydrochloric acid solution having a mass concentration of 0.2 to 0.5%, preferably water or an aqueous hydrochloric acid solution having a mass concentration of 0.2 to 0.5%, and more preferably water.

When the extraction solvent is water, a method of extraction includes water bath extraction. The water bath extraction of the present disclosure is performed at a temperature of preferably 60 to 90° C., more preferably 90° C.; the water bath extraction is preferably performed for 1 to 3 h, more preferably 2 h.

When the extraction solvent is methanol, a method of extraction includes ultrasonic extraction. The ultrasonic extraction of the present disclosure is performed at a frequency of preferably 60 to 100 Hz, for preferably 15 to 30 min; and

when the extraction solvent is an aqueous hydrochloric acid solution containing hydrochloric acid having a mass concentration of 0.2 to 0.5%, the extraction includes: mixing the Areca catechu raw material with an aqueous hydrochloric acid solution containing hydrochloric acid having a mass concentration of 0.2 to 0.5%, performing soaking for 1 h, loading the soaked mixture onto a percolation column, and performing percolation extraction with an aqueous hydrochloric acid solution containing hydrochloric acid having a mass concentration of 0.2 to 0.5%.

The present disclosure also provides a biological feed for preventing and controlling ASF, consisting of an Areca catechu product and basic feed; and
the basic feed includes any one or more of complete feed, concentrated feed and premixed feed.

In some embodiments, the Areca catechu product includes Areca catechu extract and/or Areca catechu ultrafine powder;
a content of polyphenol in the Areca catechu extract is 2% to 30%;
when the Areca catechu product is Areca catechu extract, 25 g to 400 g of the Areca catechu extract is added per 1 ton of complete feed;
when the Areca catechu product is Areca catechu ultrafine powder, 125 g to 2,000 g of the Areca catechu ultrafine powder is added per 1 ton of complete feed;
when the Areca catechu product is Areca catechu ultrafine powder and Areca catechu extract, 60 to 1,000 g of the Areca catechu ultrafine powder and 10 to 200 g of the Areca catechu extract are added per 1 ton of complete feed.

The present disclosure also provides a drinking preparation for preventing and controlling ASF, consisting of an Areca catechu product and water.

In some embodiments, the Areca catechu product includes Areca catechu extract and/or Areca catechu ultrafine powder;
polyphenol in the Areca catechu extract has a content of 2% to 30%;
when the Areca catechu product is Areca catechu extract, 25 g to 400 g of the Areca catechu extract is added per 1 ton of water;
when the Areca catechu product is Areca catechu ultrafine powder, 125 g to 2,000 g of the Areca catechu ultrafine powder is added per 1 ton of water; and
when the Areca catechu product is Areca catechu ultrafine powder and Areca catechu extract, 60 to 1,000 g of the Areca catechu ultrafine powder and 10 to 200 g of the Areca catechu extract are added per 1 ton of water.

### Beneficial Effects

In the present disclosure, the conditions of feed and drinking water in pig farms are simulated, the Areca catechu product is mixed with an ASFV suspension, the resulting mixture is then inoculated in cells, and in vitro cell experiments are carried out for evaluation. It has been found that the Areca catechu product as the sole active ingredient has a good killing effect on ASFV, where 100% of ASFV with virus titer of 5×10³ HAD₅₀/mL can be killed within 30 minutes, and 100% of ASFV with virus titer of 5×10⁴ HAD₅₀/mL can be killed within 2 hours. The use of the Areca catechu product as an additive for self-prepared feed, and disinfection of drinking water and environmental in feed industry enterprises or farms does not require the addition of expensive high-temperature granulating equipment and modifications of water supply facilities. The application is convenient, and may effectively cut off both the feed-borne and drinking water-borne transmissions of ASFV, providing important biosafety measures for the prevention and control of ASF.

### BRIEF DESCRIPTION OF THE DRAWINGS

To describe the embodiments of the present disclosure or those in the prior art more clearly, the accompanying drawings required for the examples will be briefly described below.

FIG. 1 shows a positive control of hemabsorption (HAD) detection, specifically showing the HAD detection results of group V in Example 7 where cells were directly incubated with the virus stock solution;

FIG. 2 shows a negative control of HAD detection, specifically showing the HAD detection results of group VII in Example 7 where the cells were incubated for 2 h with PBS solution;

FIG. 3 shows HAD detection results of group III in Example 7 where cells were incubated for 2 h with the virus mixture that was inactivated at 4° C. for 2 h with Areca catechu extract BL-5 having a concentration of 50 ppm (actual working concentration of 25 ppm after mixing);

FIG. 4 shows a HAD detection results of group IV in Example 7 where the cells were incubated for 2 h with virus mixture that was inactivated at 4° C. for 2 h with Areca catechu extract BL-5 having a concentration of 50 ppm (actual working concentration of 25 ppm after mixing);

FIG. 5 shows a positive control for immunofluorescence assay (IFA), specifically showing the IFA results of group V in Example 7 where the cells were incubated with the virus stock solution;

FIG. 6 shows a negative control for IFA, specifically showing the IFA results of group VII in Example 7 where the cells were incubated for 2 h with PBS solution;

FIG. 7 shows the IFA results of group III in Example 7 where the cells were incubated for 2 h with virus mixture that was inactivated at 4° C. for 2 h with Areca catechu extract BL-5 having a concentration of 50 ppm (actual working concentration of 25 ppm after mixing); and

FIG. 8 shows the IFA results of group IV in Example 7 where the cells were incubated for 2 h with virus mixture that was inactivated at 4° C. for 2 h with Areca catechu extract BL-5 having a concentration of 50 ppm (actual working concentration of 25 ppm after mixing);

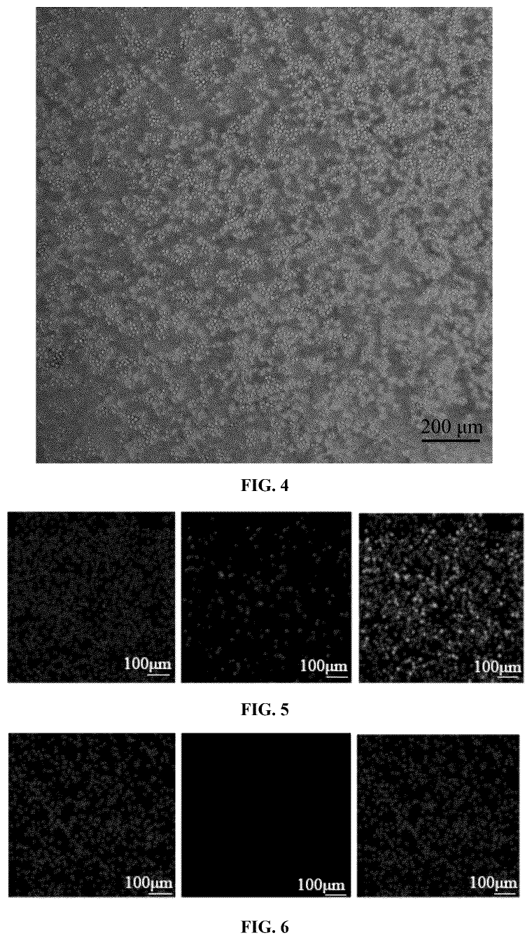

where the images in each of FIGS. 5 to 8, from left to right, sequentially show the DAPI staining results, the fluorescence staining results of ASFV P30 protein, and the merge image of DAPI staining and fluorescence staining, with a scale bar of 100 μm.

### DETAILED DESCRIPTION OF THE EMBODIMENTS

The present disclosure provides use of an Areca catechu product as a sole active ingredient in manufacturing a biological feed and/or a dinking preparation for preventing and controlling African swine fever.

In the present disclosure, the Areca catechu product preferably includes Areca catechu extract and/or Areca catechu ultrafine powder, more preferably an Areca catechu extract. The Areca catechu extract of the present disclosure is preferably a water extract of Areca catechu; the content of polyphenol in the Areca catechu extract is preferably 2% to 30%, more preferably 8.8% to 30%, still preferably 10.72% to 24.01%, more preferably 13.67% to 19.56%, most preferably 18.80%; the Areca catechu ultrafine powder is preferably the undersize of a sieve with meshes of ≥80.

In the present disclosure, there is no strict requirement on the manner in which the Areca catechu product is added to a feed or water, that is, the Areca catechu product can be added to a feed or daily drinking water in the form of a premix or a powder.

In the present disclosure, there is no strict requirement on the source of the Areca catechu extract or the Areca catechu ultrafine powder, that is, the Areca catechu extract or the Areca catechu ultrafine powder can be purchased directly or prepared independently. In the present disclosure, an independent preparation method is preferably used to obtain the Areca catechu extract. In the present disclosure, the method for preparing the Areca catechu extract preferably includes the steps of:
mixing an Areca catechu raw material with an extraction solvent, performing extraction, and collecting a liquid extract of Areca catechu;
and subjecting the liquid extract of Areca catechu to drying to obtain the Areca catechu extract.

In the present disclosure, an Areca catechu raw material is mixed with an extraction solvent, extraction is performed, and a liquid extract of Areca catechu is collected. In the present disclosure, the Areca catechu raw material preferably includes fresh fruits of Areca catechu and/or dried fruits of Areca catechu; in the present disclosure, there is no strict requirement on the maturity of the fresh fruits of Areca catechu or the dried fruits of Areca catechu, that is, the fresh fruits of Areca catechu or the dried fruits of Areca catechu with any maturity can be used; in the present disclosure, the mass-to-volume ratio of the Areca catechu raw material to the extraction solvent is preferably 1 g:(8 to 35) mL, more preferably 1 g:(10 to 20) mL, even more preferably 1 g:(15 to 20) mL, and most preferably 1 g:20 mL. In the present disclosure, a crusher is preferably used to crush the Areca catechu raw material, and the crushed Areca catechu is mixed with water for extraction. In the present disclosure, the method of collecting the liquid extract of Areca catechu is preferably filtration.

In the present disclosure, the extraction solvent is preferably any one of water, methanol, and an aqueous hydrochloric acid solution having a mass concentration of 0.2 to 0.5%, more preferably water or an aqueous hydrochloric acid solution having a mass concentration of 0.2 to 0.5%, and even more preferably water. In the present disclosure, methanol having a volume concentration of 50% is preferable.

In the present disclosure, when the extraction solvent is water, the extraction preferably includes water bath extraction; the water bath extraction is preferably performed at a temperature from 60 to 90° C., more preferably 90° C.; the water bath extraction is preferably performed for 1 to 3 h, more preferably for 2 h.

In the present disclosure, when the extraction solvent is methanol, the ultrasonic extraction is preferably performed at a frequency of 60 to 100 Hz, more preferably 70 Hz, for preferably 15 to 30 minutes, more preferably 20 minutes.

In the present disclosure, when the extraction solvent is an aqueous hydrochloric acid solution having a mass concentration of 0.2 to 0.5%, the extraction includes: mixing the Areca catechu raw material with an aqueous hydrochloric acid solution having a mass concentration of 0.2 to 0.5%, performing soaking for 1 h, loading the soaked mixture onto a percolation column, and performing percolation extraction with an aqueous hydrochloric acid solution having a mass concentration of 0.2 to 0.5%.

In the present disclosure, after the liquid extract of Areca catechu is obtained, the Areca catechu concentrate is preferably subjected to drying. In the present disclosure, the drying is preferably at a temperature from 65 to 75° C., more preferably 70° C., and the drying time is preferably 72 to 96 h, more preferably 75 to 90 h, and even more preferably 80 to 85 h.

In the present disclosure, before the liquid extract of Areca catechu is dried, it is preferably subjected to concentration, and the concentration in vacuum/under reduced pressure is the first choice. In the present disclosure, the cycle of concentration is preferably 30 to 35, more preferably 32. In the present disclosure, there is no strict requirement on the specific parameter conditions of the concentration, as long as the stock solution could be concentrated into 1/30 to 1/35 of the volume of the filtrate. In the present disclosure, when the extraction solvent is aqueous hydrochloric acid solution having a mass concentration of 0.2 to 0.5%, and the liquid extract of Areca catechu preferably passes through a 732 cation adsorption column to obtain the effluent from the cation adsorption column before concentration treatment.

In the present disclosure, the situation where feeds and drinking water in pig industry are contaminated by high-load virus is simulated. The Areca catechu product is subjected to ASFV inactivation experiment as the sole active ingredient, and the results showed that the Areca catechu product can have a good killing effect on ASFV.

The present disclosure also provides a biological feed for preventing and controlling African swine fever, consisting of an Areca catechu product and basic feed; and the basic feed includes any one or more of a complete feed, a concentrated feed and a premixed feed.

In the present disclosure, the basic feed includes any one or more of a complete feed, a concentrated feed and a premixed feed, more preferably any one of a complete feed, a concentrated feed and a premixed feed.

In the present disclosure, when the basic feed is a complete feed, the Areca catechu product is preferably added directly to the complete feed to obtain the biological feed. In the present disclosure, the Areca catechu product preferably includes Areca catechu extract and/or Areca catechu ultrafine powder. In the present disclosure, when the Areca catechu product is an Areca catechu extract, preferably 25 g to 400 g of the Areca catechu extract is added per 1 ton of complete feed, more preferably 125 g to 300 g, even more preferably 150 g to 200 g. In the present disclosure, when the Areca catechu extract is added, preferably any value in the range of 25 g to 400 g could be selected, such as 25 g, 28 g, 30 g, 40 g, 50 g, 55 g, 60 g, 80 g, 100 g, 125 g, 130 g, 145 g, 150 g, 180 g, 195 g, 200 g, 230 g, 250 g, 260 g, 280 g, 300 g, 350 g, 360 g, 380 g or 400 g. In the present disclosure, when the Areca catechu product is Areca catechu ultrafine powder, preferably 125 g to 2,000 g of the Areca catechu ultrafine powder is added per 1 ton of complete feed, more preferably 250 g to 1,500 g, even more preferably 400 g to 1,200 g, most preferably 800 g to 1,200 g. In the present disclosure, when the Areca catechu ultrafine powder is added, preferably any value in the range of 125 g to 2,000 g is selected. In the present disclosure, when the Areca catechu product is an Areca catechu extract and Areca catechu ultrafine powder, preferably 60 to 1,000 g of the Areca catechu ultrafine powder and 10 to 200 g of the Areca catechu extract are added per 1 ton of complete feed. The additive amount of the Areca catechu ultrafine powder is preferably at 100 to 800 g, more preferably 200 to 600 g, and even more preferably 400 to 600 g per ton of complete feed; the additive amount of the Areca catechu extract is preferably 40 to 180 g, more preferably 60 to 140 g, and even more preferably 80 to 140 g or 120 g per ton of complete feed.

In the present disclosure, when the basic feed is a concentrated feed, the Areca catechu product is added to the concentrated feed preferably according to the concentration multiple of concentration of the concentrated feed and the mass of the Areca catechu product that needs to be added to the complete feed described above to obtain the biological feed. When the multiple of concentration is N, and X g of the Areca catechu product needs to be added to 1 ton of complete feed, NX g of the Areca catechu product is added per ton of concentrated feed to obtain the biological feed. In the present disclosure, the concentration is preferably conducted for 3 to 5 multiples. The biological feed of the present disclosure may be sold separately, or be mixed with other ingredients to prepare a complete feed for sale, such as an energy feed. In the present disclosure, when a complete feed is prepared, X g of the Areca catechu product is preferably added per ton of complete feed.

In the present disclosure, when the basic feed is a premixed feed, the Areca catechu product is added to the premixed feed preferably according to the multiple of concentration of the premixed feed and the mass of the Areca catechu product that needs to be added to the complete feed described above to obtain the biological feed. When the concentration multiple of the premixed feed is N, and X g of the Areca catechu product needs to be added to 1 ton of complete feed, and NX g of the Areca catechu product is added per ton of the premixed feed to obtain the biological feed. In the present disclosure, the multiple of concentration is preferably 20 to 25. The biological feed of the present disclosure may be sold separately, or be mixed with other ingredients to prepare a complete feed for sale. In the present disclosure, when a complete feed is prepared, preferably X g of the Areca catechu product is added per ton of complete feed.

In the present disclosure, there is no strict requirement on the sources of the complete feed, concentrated feed and premixed feed, that is, the complete feed, concentrated feed and premixed feed can be purchased conventionally or prepared independently.

The present disclosure preferably provides a method for preparing the above biological feed, which preferably includes the steps of: mixing the Areca catechu product with basic feed, and leaving the resulting mixture to stand for 0.5 h to 3 h to obtain the feed, more preferably to stand for 1 to 2 h.

In the present disclosure, the Areca catechu product is added to the basic feed, which can kill ASFV that may cause contamination in the feed, block the feed-borne transmission of ASFV, and prevent and control African swine fever.

The present disclosure also provides a dinking preparation for preventing and controlling African swine fever, consisting of an Areca catechu product and water.

In the present disclosure, the Areca catechu product preferably includes an Areca catechu extract and/or Areca catechu ultrafine powder. In the present disclosure, when the Areca catechu product is Areca catechu extract, 1 ton of water need to add to 25 g to 400 g of the Areca catechu extract, preferably 125 g to 200 g, more preferably 150 g. In the present disclosure, when the Areca catechu extract is added, any value in the range of preferably 25 g to 400 g is selected, such as 25 g, 28 g, 30 g, 40 g, 50 g, 55 g, 60 g, 80 g, 100 g, 125 g, 130 g, 145 g, 150 g, 180 g, 195 g, 200 g, 230 g, 250 g, 260 g, 280 g, 300 g, 350 g, 360 g, 380 g or 400 g.

In the present disclosure, when the Areca catechu product is Areca catechu ultrafine powder, 1 ton of water need to add to 125 g to 2,000 g of the Areca catechu ultrafine powder, preferably 250 g to 1,500 g, more preferably 400 g to 1,200 g, most preferably 800 g to 1,200 g. In the present disclosure, when the Areca catechu ultrafine powder is added, any value in the range of preferably 125 g to 2,000 g is selected.

In the present disclosure, when the Areca catechu product is Areca catechu extract and Areca catechu ultrafine powder, 60 to 1,000 g of the Areca catechu ultrafine powder and 10 to 200 g of the Areca catechu extract are preferably added per 1 ton of water. Preferably, 100 to 800 g, more preferably 200 to 600 g, and even more preferably 400 to 600 g of the Areca catechu ultrafine powder is added per ton of drinking water; the additive amount of the Areca catechu extract is preferably 40 to 180 g, more preferably 60 to 140 g, even more preferably 80 to 140 g or 120 g per ton of drinking water.

The present disclosure preferably provides a method for preparing the above dinking preparation, which preferably includes the steps of: mixing Areca catechu extract with water, and leaving the resulting mixture to stand for 0.5 h to 3 h to obtain the dinking preparation, more preferably for 1 to 2 h; alternatively, the method preferably includes the steps of: putting Areca catechu ultrafine powder in a filter bag, and soaking the filter bag in water for 12 h or more to obtain the dinking preparation.

In the present disclosure, the Areca catechu product is added to water, so that a resulting liquid preparation can be used directly as drinking water for animals, which kills ASFV that may cause contamination in the drinking water, blocks the water-borne transmission of ASFV, and prevents and controls African swine fever; and the liquid preparation can also be sprayed in the pigsty environment, which blocks the environment-borne transmission of ASFV and prevents and controls African swine fever.

To further illustrate the present disclosure, the use of the Areca catechu product provided by the present disclosure as a sole active ingredient in manufacturing a biological feed and/or a dinking preparation for preventing and controlling African swine fever is described in detail below in conjunction with the accompanying drawings and examples, but they cannot be construed as limiting the scope of protection of the present disclosure.

### Example 1

The Areca catechu extract was prepared according to the following steps:
(1) decoction pieces of Areca catechu were crushed and the crushed Areca catechu was obtained;
(2) the crushed Areca catechu and 50% methanol were mixed with a mass-to-volume ratio of 1 g:20 mL, and a resulting mixture was ultrasonicated at 70 Hz for 20 min to obtain an ultrasonic liquid extract, which was then filtered to obtain a liquid extract of Areca catechu;
(3) the liquid extract of Areca catechu was concentrated to 1/30 of the volume of the filtrate using concentration in vacuum/under reduced pressure, and then dried at 70° C. for 72 to 96 h to obtain the Areca catechu extract.

### Example 2

The Areca catechu extract was prepared according to the following steps:
(1) dried fruits of Areca catechu were crushed and the crushed Areca catechu was obtained;
(2) the crushed Areca catechu and water were mixed with a mass-to-volume ratio of 1 g:25 mL, and a resulting mixture was extracted for 1 h under a water bath condition at 80° C., and then filtered to obtain a liquid extract of Areca catechu;
(3) the liquid extract of Areca catechu was concentrated to 1/35 of the volume of the filtrate by using concentration in vacuum/under reduced pressure, and then dried at 70° C. for 72 to 96 h to obtain the Areca catechu extract.

### Example 3

The Areca catechu extract was prepared according to the following steps:
(1) dried fruits of Areca catechu were crushed and the crushed Areca catechu was obtained;
(2) the crushed Areca catechu and an aqueous hydrochloric acid solution having a mass concentration of 0.5% were mixed with a mass-to-volume ratio of 1 g:10 mL, and soaked for 1 h before loading onto a percolation column, and percolation extraction was performed with 3,000 mL of an aqueous hydrochloric acid solution having a mass concentration of 0.5% to obtain the hydrochloric acid filtrate of Areca catechu;
(3) the hydrochloric acid filtrate of Areca catechu was passed through a 400 mL 732 cation adsorption column to obtain the effluent from the cation adsorption column;
(4) the pH value of the effluent was adjusted to 7 with a 1% sodium hydroxide solution, the effluent was concentrated to 1/40 of the total volume using concentration in vacuum/under reduced pressure, and then dried at 70° C. for 72 to 96 h to obtain the Areca catechu extract.

### Example 4

The steps were the same with Example 2 except that in step (2), the crushed Areca catechu and water were mixed with a mass-to-volume ratio of 1 g:15 mL.

### Example 5

The steps were the same with Example 3 except that in step (2), the crushed Areca catechu and an aqueous hydrochloric acid solution having a mass concentration of 0.2% were mixed with a mass-to-volume ratio of 1 g:10 mL, and the mixtures were loaded onto a percolation column after soaked for 1 h, and percolation extraction was performed with 3,000 mL of an aqueous hydrochloric acid solution having a mass concentration of 0.2% to obtain a hydrochloric acid filtrate of Areca catechu.

### Test Example 1

The content of the ingredients in the Areca catechu extracts obtained in Examples 1 to 5 was analyzed using liquid chromatography and folin-phenol method. The results are as shown in Table 1.

TABLE 1
Detection results of ingredients in the Areca catechu
extracts obtained in Examples 1 to 5 and content thereof

| | | |
|---|---|---|
| | Total alkali | Total phenol |
| Sample number | content (%) | content (%) |

| Example 1 | | | 0.57 | 10.72 |
|---|---|---|---|---|
| | Example 2 | Replicate 1 | 1.54 | 19.32 |
| | | Replicate 2 | 1.58 | 19.84 |
| | | Replicate 3 | 1.59 | 19.52 |
| | | Mean value | 1.57 | 19.56 |
| | Example 3 | Replicate 1 | 0.042 | 19.30 |
| | | Replicate 2 | 0.045 | 18.74 |
| | | Replicate 3 | 0.045 | 18.37 |
| | | Mean value | 0.044 | 18.80 |
| | Example 4 | Replicate 1 | 1.56 | 14 |
| | | Replicate 2 | 1.11 | 14 |
| | | Replicate 3 | 1.69 | 13 |
| | | Mean value | 1.45 | 13.67 |

| | | |
|---|---|---|
| Example 5 | 1.32 | 24.01 |

It can be seen from Table 1 that in the Areca catechu extracts obtained in the present disclosure, the total phenol content was 10.72% to 24.01% and the total alkali content was 0.044% to 1.57%.

### Example 6

The situation where feeds and drinking water in pig industry are contaminated by high-load virus was simulated. With water as a solvent, the Areca catechu extract prepared in Example 1 was prepared into extracts having three concentrations (400 parts per million (ppm), 800 ppm and 1,600 ppm), respectively, where 1 ppm meant adding 1 mg of the Areca catechu extract to 1 L of water.

In cooperation with the African Swine Fever Regional Laboratory of China (Guangzhou), we conducted an ASFV inactivation experiment using the Areca catechu extracts, where the concentration of the ASFV in this experiment was 10⁴ HAD₅₀/mL.

The specific experimental steps were as follows:
(1) experiments were performed in groups: the Areca catechu extract reacted with the viral inoculums, with 4 replicates for each group.
Group I: at 4° C., the Areca catechu extract and the viral inoculums (10⁴ HAD₅₀/mL) were respectively mixed for 30 min, 1 h, 2 h and 3 h to obtain samples with different virus inactivation times, each of which was diluted 100 times, respectively, and then inoculated into cells.
Group II: at 25° C., the Areca catechu extract and the viral inoculums (10⁴ HAD₅₀/mL) were respectively mixed for 30 min, 1 h, 2 h and 3 h to obtain samples with different virus inactivation times, each of which was diluted 100 times, respectively, and then inoculated into cells.
Group III: at 4° C., the viral inoculums (10⁴ HAD₅₀/mL) was diluted 10 times, 100 times, 1,000 times and 10,000 times to obtain various concentrations of viral inoculums which were then inoculated into the cells.
Group IV: at 25° C., the viral inoculums (10⁴ HAD₅₀/mL) was diluted 10 times, 100 times, 1,000 times and 10,000 times to obtain various concentrations of viral inoculums, which were then inoculated into the cells.
Group V: at 4° C., the phosphate buffered saline (PBS) solution was inoculated into cells.
Group VI: in an environment of 25° C., the PBS solution was inoculated into cells.
(2) Porcine alveolar macrophage (PAM) cells were plated in 96-well plates. In group I, 0.5 mL of the Areca catechu extracts having the concentrations of 400 ppm, 800 ppm and 1,600 ppm were taken and reacted with 0.5 mL of the viral inoculums (10⁴ HAD₅₀/mL) at 4° C. for the predetermined virus inactivation time (i.e., reaction for 30 min, 1 h, 2 h, 3 h). Following this, the mixtures were diluted 100 times; and the diluted mixtures were inoculated into 96-well plates containing PAM cells according to an inoculating amount of 200 μL/well and incubated for 2 h. After incubation, the mixtures were removed, and fresh cell maintenance medium was added. The plates were placed in an incubator at 37° C. and 5% CO₂ for culture.

In group II, 0.5 mL of the Areca catechu extracts having the concentrations of 400 ppm, 800 ppm and 1,600 ppm were taken and reacted with 0.5 mL of the viral inoculums (10⁴ HAD₅₀/mL) at 25° C. for the predetermined virus inactivation time (i.e., reaction for 30 min, 1 h, 2 h, 3 h). The mixtures were diluted 100 times; and the diluted mixtures were inoculated into 96-well plates containing PAM cells according to an inoculating amount of 200 μL/well, and incubated for 2 h. After incubation, the mixtures were removed, and fresh cell maintenance medium was added, The plates were placed in an incubator at 37° C. and 5% CO₂ for culture.

In group III, the different concentrations of viral inoculums were inoculated into 96-well plates containing PAM cells according to an inoculating amount of 200 μL/well at 4° C., respectively, and incubated for 2 h. After incubation, the mixtures were removed, and fresh cell maintenance medium was added. The plates were placed in an incubator at 37° C. and 5% CO₂ for culture.

In group IV, the different concentrations of viral inoculums were inoculated into 96-well plates containing PAM cells according to an inoculating amount of 200 μL/well at 25° C., respectively, and then incubated for 2 h. After incubation, the mixtures were removed, and fresh cell maintenance medium was added. The plates were placed in an incubator at 37° C. and 5% CO₂ for culture.

In group V, the PBS solution was inoculated into 96-well plates containing PAM cells according to an inoculating amount of 200 μL/well at 4° C., and incubated for 2 h. After incubation, the PBS solution was removed, and fresh cell maintenance medium was added. The plates were placed in an incubator at 37° C. and 5% CO₂ for culture.

In group VI, the PBS solution was inoculated into 96-well plates containing PAM cells according to an inoculating amount of 200 μL/well at 25° C., and then incubated for 2 h. After incubation, the PBS solution was removed, a cell maintenance medium was additionally added, and the plates were placed in an incubator at 37° C. and 5% CO₂ for culture.

The above cell maintenance medium consisted of 10% fetal bovine serum, 100 U·mL⁻¹ penicillin, 100 U·mL⁻¹ streptomycin and the balance of RPMI-1640 medium, where the fetal bovine serum (FBS), RPMI-1640 medium, penicillin (cell culture grade), and streptomycin (cell culture grade) were all purchased from Gibco.
(3) After 48 h of culture in step (2), the PAM cells were seeded into 96-well plates and cultured until 70%˜80% confluence, and then hemadsorption (HAD) test was performed by the African Swine Fever Regional Laboratory of China (Guangzhou). The results are shown in Table 2.

TABLE 2
Results of hemadsorption (HAD) test

| | Concentration of | | | | | | |
|---|---|---|---|---|---|---|---|
| Treated | Areca catechu | | | Reaction time | | | |
| groups | extract | | Viral inoculums | 30 min | 1 h | 2 h | 3 h |
| Group I | 400 | ppm | 10⁴ HAD₅₀/mL | − | − | − | − |
| | 800 | ppm | 10⁴ HAD₅₀/mL | − | − | − | − |
| | 1,600 | ppm | 10⁴ HAD₅₀/mL | − | − | − | − |
| Group II | 400 | ppm | 10⁴ HAD₅₀/mL | − | − | − | − |
| | 800 | ppm | 10⁴ HAD₅₀/mL | − | − | − | − |
| | 1,600 | ppm | 10⁴ HAD₅₀/mL | − | − | − | − |

| | | | | | | |
|---|---|---|---|---|---|---|
| Group III | / | 10⁴ HAD₅₀/mL | + | + | + | + |
| | / | 10³ HAD₅₀/mL | + | + | + | + |
| | / | 10² HAD₅₀/mL | + | + | + | + |
| | / | 10¹ HAD₅₀/mL | + | + | + | + |
| | / | 10⁰ HAD₅₀/mL | + | + | + | + |
| Group IV | / | 10⁴ HAD₅₀/mL | + | + | + | + |
| | / | 10³ HAD₅₀/mL | + | + | + | + |
| | / | 10² HAD₅₀/mL | + | + | + | + |
| | / | 10¹ HAD₅₀/mL | + | + | + | + |
| | / | 10⁰ HAD₅₀/mL | + | + | + | + |
| Group V | / | / | − | − | − | − |
| Group VI | / | / | − | − | − | − |

Notes:
“−” and “+” indicate the results of HAD test are negative and positive, respectively.

It can be seen from Table 2 that the Areca catechu extract having the lowest actual working concentration of 200 ppm could effectively inactivates ASFV with a virus titer of 5×10³ HAD₅₀/mL within 30 minutes, and the result of hemadsorption (HAD) test was negative for the virus.

### Example 7

The situation where feeds and drinking water in pig industry are contaminated by high-load virus was simulated. With water as a solvent, the Areca catechu extract prepared in Example 2 (designated as BL-1) was prepared into extracts having three concentrations (400 ppm, 200 ppm and 100 ppm), respectively, and the Areca catechu extract prepared in Example 3 (designated as BL-5) was prepared into extracts having three concentrations (200 ppm, 100 ppm and 50 ppm), respectively. In cooperation with the African Swine Fever Regional Laboratory of China (Guangzhou), we conducted an ASFV inactivation experiment using the Areca catechu extracts, where the concentration of the ASFV in this experiment was 10⁵ HAD₅₀/mL.

The specific experimental steps were as follows:
(1) experiments were performed in groups: the Areca catechu extract reacted with the viral inoculums, with 4 repeated experiments for each group.
Group I: at 4° C., the Areca catechu extract BL-1 and the viral inoculums (10⁵ HAD₅₀/mL) were mixed for 2 h, diluted 100 times, and inoculated into cells.
Group II: at 25° C., the Areca catechu extract BL-1 and the viral inoculums (105 HAD₅₀/mL) were mixed for 2 h, diluted 100 times, and inoculated into cells.
Group III: at 4° C., the Areca catechu extract BL-5 and the viral inoculums (105 HAD₅₀/mL) were mixed for 2 h, diluted 100 times, and inoculated into cells.
Group IV: at 25° C., the Areca catechu extract BL-5 and the viral inoculums (105 HAD₅₀/mL) were mixed for 2 h, diluted 100 times, and inoculated into cells.
Group V: at 4° C., the viral inoculums (10⁵ HAD₅₀/mL), as well as dilutions at 10 times, 100 times, 1,000 times, and 10,000 times were inoculated into cells, respectively.
Group VI: at 25° C., the viral inoculums (10⁵ HAD₅₀/mL), as well as dilutions at 10 times, 100 times, 1,000 times, and 10,000 times were inoculated into cells, respectively.
Group VII: at 4° C., a PBS solution was inoculated into cells.
Group VIII: at 25° C., a PBS solution was inoculated into cells.
(2) PAM cells were plated in 96-well plates. In group I, 0.5 mL of the Areca catechu extracts having different concentrations were taken and reacted with 0.5 mL of the Viral inoculums (10⁵ HAD₅₀/mL) at 4° C. for the predetermined virus inactivation time (i.e., 2 h). The mixtures were diluted 100 times; and then inoculated into 96-well plates containing PAM cells according to an inoculating amount of 200 μL/well, followed by incubation for 2 h. After the incubation, the mixtures were removed, and fresh cell maintenance medium was added. The plates were placed in an incubator at 37° C. and 5% CO₂ for culture.

In group II, 0.5 mL of the Areca catechu extracts having different concentrations were taken and reacted with 0.5 mL of the viral inoculums (10⁵ HAD₅₀/mL) at 25° C. for the predetermined virus inactivation time (i.e., reaction for 2 h). The mixtures were diluted 100 times, and then inoculated into 96-well plates containing PAM cells according to an inoculating amount of 200 μL/well, followed by incubation for 2 h. After the incubation, the mixtures were removed, and fresh cell maintenance medium was added. The plates were placed in an incubator at 37° C. and 5% CO₂ for culture.

In group III, 0.5 mL of the Areca catechu extracts having different concentrations were taken and reacted with 0.5 mL of the Viral inoculums (10⁵ HAD₅₀/mL) at 4° C. for the predetermined virus inactivation time (i.e., 2 h). The mixtures were diluted 100 times, and then inoculated into 96-well plates containing PAM cells according to an inoculating amount of 200 μL/well, followed by incubation for 2 h. After the incubation, the mixtures were removed, and fresh cell maintenance medium was added. The plates were placed in an incubator at 37° C. and 5% CO₂ for culture.

In group IV, 0.5 mL of the Areca catechu extracts having different concentrations were taken and reacted with 0.5 mL of the viral inoculums (10⁵ HAD₅₀/mL) at 25° C. for the predetermined virus inactivation time (i.e., reaction for 2 h). The mixtures were diluted 100 times, and then inoculated into 96-well plates containing PAM cells according to an inoculating amount of 200 μL/well, followed by incubation for 2 h. The mixtures were removed, and fresh cell maintenance medium was added. The plates were placed in an incubator at 37° C. and 5% CO₂ for culture.

In group V, the different concentrations of viral inoculums were inoculated into 96-well plates containing PAM cells according to an inoculating amount of 200 μL/well at 4° C., and then incubated for 2 h. After incubation, the mixtures were removed, and fresh cell maintenance medium was added. The plates were placed in an incubator at 37° C. and 5% CO₂ for culture.

In group VI, the different concentrations of viral inoculums were inoculated into 96-well plates containing PAM cells according to an inoculating amount of 200 μL/well at 25° C., and incubated for 2 h. After incubation, the mixtures were removed, and fresh cell maintenance medium was added. The plates were placed in an incubator at 37° C. and 5% CO₂ for culture.

In group VII, the PBS solution was inoculated into 96-well plates containing PAM cells according to an inoculating amount of 200 μL/well at 4° C., and then incubated for 2 h. After incubation, the PBS solution was removed, and fresh cell maintenance medium was added. The plates were placed in an incubator at 37° C. and 5% CO₂ for culture.

In group VIII, the PBS solution was inoculated into 96-well plates containing PAM cells according to an inoculating amount of 200 μL/well at 25° C., and then incubated for 2 h. After incubation, the PBS solution was removed, and fresh cell maintenance medium was added. The plates were placed in an incubator at 37° C. and 5% CO₂ for culture.

The cell maintenance medium consisted of 10% fetal bovine serum, 100 U·mL⁻¹ penicillin, 100 U·mL⁻¹ streptomycin and the balance of RPMI-1640 medium, where the fetal bovine serum (FBS), RPMI-1640 medium, penicillin (cell culture grade), and streptomycin (cell culture grade) were all purchased from Gibco.
(3) After 48 h of culture in step (2), the PAM cells were seeded into 96-well plates and cultured until 70%˜80% confluence, and then hemadsorption (HAD) test and indirect immunofluorescence assay (IFA) were performed by the African Swine Fever Regional Laboratory of China (Guangzhou). The results are shown in Table 3.

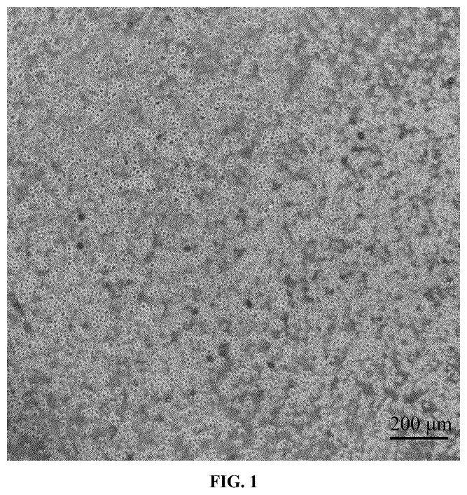

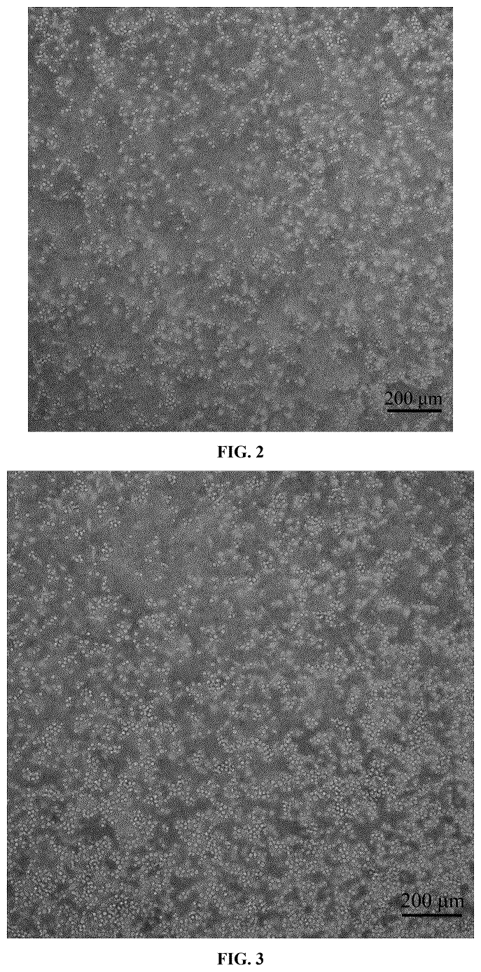

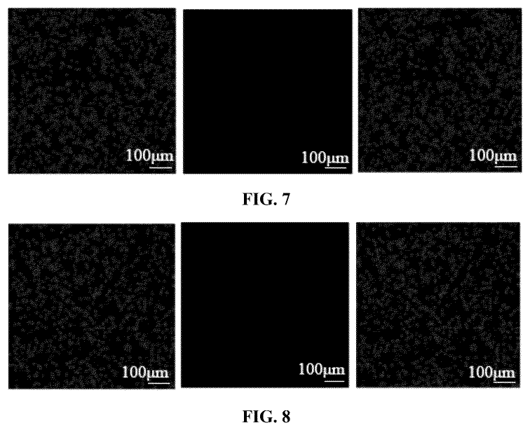

Due to the similar results between the treatment with three concentrations of BL-1 and the results of the treatment with three concentrations of BL-5 at different temperatures in the experimental groups and the excessive grouping, and considering the size of the document, only part of HAD detection results (as shown in FIGS. 1 to 4) and IFA results (as shown in FIGS. 5 to 8) are provided. FIGS. 1 and 5 show positive controls, specifically showing the detection results of group V, where the cells were directly incubated with the ASFV (4° C., no Areca catechu extract); FIG. 2 and FIG. 6 show negative controls, specifically showing the results of group VII, where the cells were incubated with the PBS solution for 2 h (4° C., no virus); FIG. 3 and FIG. 7 show the detection results of group III, where the virus was inactivated for 2 h with the Areca catechu extract BL-5 having a concentration of 50 ppm (actual working concentration of 25 ppm after mixing) and then incubated with cells for 2 h (4° C.-2 h inactivation); FIG. 4 and FIG. 8 specifically show the detection results of group IV, where the ASFV was inactivated for 2 h with the Areca catechu extract BL-5 having a concentration of 50 ppm (actual working concentration of 25 ppm after mixing) and then incubated with cells for 2 h (25° C.-2 h inactivation). The images in each of FIGS. 5 to 8, from left to right, sequentially show the DAPI staining results, the fluorescence staining results of ASFV P30 protein, and the merge image of DAPI staining and fluorescence staining.

TABLE 3
Results of hemadsorption (HAD)

| | Concentration of | | |
|---|---|---|---|
| Treated | Areca catechu | Viral | Reaction |
| groups | extract | inoculums | time (2 h) |
| Group I | 400 ppm | 10⁵ HAD₅₀/mL | − |
| | 200 ppm | 10⁵ HAD₅₀/mL | − |
| | 100 ppm | 10⁵ HAD₅₀/mL | − |
| Group II | 400 ppm | 10⁵ HAD₅₀/mL | − |
| | 200 ppm | 10⁵ HAD₅₀/mL | − |
| | 100 ppm | 10⁵ HAD₅₀/mL | − |
| Group III | 200 ppm | 10⁵ HAD₅₀/mL | − |
| | 100 ppm | 10⁵ HAD₅₀/mL | − |
| | 50 ppm | 10⁵ HAD₅₀/mL | − |
| Group IV | 200 ppm | 10⁵ HAD₅₀/mL | − |
| | 100 ppm | 10⁵ HAD₅₀/mL | − |
| | 50 ppm | 10⁵ HAD₅₀/mL | − |
| Group V | / | 10⁵ HAD₅₀/mL | + |
| | / | 10⁴ HAD₅₀/mL | + |
| | / | 10³ HAD₅₀/mL | + |
| | / | 10² HAD₅₀/mL | + |
| | / | 10¹ HAD₅₀/mL | + |
| Group VI | / | 10⁵ HAD₅₀/mL | + |
| | / | 10⁴ HAD₅₀/mL | + |
| | / | 10³ HAD₅₀/mL | + |
| | / | 10² HAD₅₀/mL | + |
| | / | 10¹ HAD₅₀/mL | + |
| Group VII | / | / | − |
| Group VIII | / | / | − |

Notes:
“−” indicates that the results of HAD test and IFA are both negative, and “+” indicates that the results of HAD test and IFA are both positive.

It can be seen from FIGS. 5 to 8 that under the treatment of group V, green fluorescence was present in the nuclei of PAM cells, indicating the presence of ASFV, while under the other treatments, no fluorescence signal was generated. In view of Table 3, it can be seen that at 4° C. and 25° C., the Areca catechu extract BL-1 having the lowest actual working concentration of 50 ppm or the Areca catechu extract BL-5 having the lowest actual working concentration of 25 ppm could inactivate 100% of ASFV with the virus titer of 5×10⁴ HAD₅₀/mL within 2 hours.

### Example 8

25 g of any one of the Areca catechu extracts in Examples 1 to 5 was added per ton of complete feed to obtain five biological feeds, specifically as follows:
biological feed 1:25 g of the Areca catechu extract in Example 1 was added per ton of complete feed;
biological feed 2:25 g of the Areca catechu extract in Example 2 was added per ton of complete feed;
biological feed 3:25 g of the Areca catechu extract in Example 3 was added per ton of complete feed;
biological feed 4:25 g of the Areca catechu extract in Example 4 was added per ton of complete feed; and
biological feed 5:25 g of the Areca catechu extract in Example 5 was added per ton of complete feed.

The following examples all adopt the same wording.

### Example 9

200 g of any one of the Areca catechu extracts in Examples 1 to 5 was added per ton of complete feed to obtain five biological feeds.

### Example 10

400 g of any one of the Areca catechu extracts in Examples 1 to 5 was added per ton of complete feed to obtain five biological feeds.

### Example 11

125 g of Areca catechu ultrafine powder (80 meshes or more) was added per ton of complete feed to obtain five biological feeds.

### Example 12

1,000 g of an Areca catechu ultrafine powder (80 meshes or more) was added per ton of complete feed to obtain five biological feeds.

### Example 13

2,000 g of an Areca catechu ultrafine powder (80 meshes or more) was added per ton of complete feed to obtain five biological feeds.

### Example 14

125 g of any of the Areca catechu extracts in Examples 1 to 5 was added per ton of concentrated feed having a cycle of concentration of 5 to obtain five biological feeds, and when the biological feed and an energy feed were mixed to prepare a complete feed, the proportion of the biological feed in the complete feed was 1/5, that is, the Areca catechu extract added per ton of complete feed was 25 g.

### Example 15

625 g of any of the Areca catechu extracts in Examples 1 to 5 was added per ton of concentrated feed having a cycle of concentration of 5 to obtain five biological feeds, and when the biological feed and an energy feed were mixed to prepare a complete feed, the proportion of the biological feed in the complete feed was 1/5, that is, the Areca catechu extract added per ton of complete feed was 125 g.

### Example 16

2,000 g of any of the Areca catechu extracts in Examples 1 to 5 was added per ton of concentrated feed having a cycle of concentration of 5 to obtain five biological feeds, and when the biological feed and an energy feed were mixed to prepare a complete feed, the proportion of the biological feed in the complete feed was 1/5, that is, the Areca catechu extract added per ton of complete feed was 400 g.

### Example 17

625 g of an Areca catechu ultrafine powder (80 meshes or more) was added per ton of concentrated feed having a cycle of concentration of 5 to obtain a biological feed, and when the biological feed and an energy feed were mixed to prepare a complete feed, the proportion of the biological feed in the complete feed was 1/5, that is, the Areca catechu ultrafine powder (80 meshes or more) added per ton of complete feed was 125 g.

### Example 18

3,125 g of an Areca catechu ultrafine powder (80 meshes or more) was added per ton of concentrated feed having a cycle of concentration of 5 to obtain a biological feed, and when the biological feed and an energy feed were mixed to prepare a complete feed, the proportion of the biological feed in the complete feed was 1/5, that is, the Areca catechu ultrafine powder (80 meshes or more) added per ton of complete feed was 625 g.

### Example 19

10,000 g of an Areca catechu ultrafine powder (80 meshes or more) was added per ton of concentrated feed having a cycle of concentration of 5 to obtain a biological feed, and when the biological feed and an energy feed were mixed to prepare a complete feed, the proportion of the biological feed in the complete feed was 1/5, that is, the Areca catechu ultrafine powder (80 meshes or more) added per ton of complete feed was 2,000 g.

### Example 20

625 g of any of the Areca catechu extracts in Examples 1 to 5 was added per ton of premixed feed having a cycle of concentration of 25 to obtain five biological feeds, and when the biological feed was mixed with an energy feed and a protein feed to prepare a complete feed, the proportion of the biological feed in the complete feed was 1/25, that is, the Areca catechu extract added per ton of complete feed was 25 g.

### Example 21

10,000 g of any of the Areca catechu extracts in Examples 1 to 5 was added per ton of premixed feed having a cycle of concentration of 25 to obtain five biological feeds, and when the biological feed was mixed with an energy feed and a protein feed to prepare a complete feed, the proportion of the biological feed in the complete feed was 1/25, that is, the Areca catechu extract added per ton of complete feed was 400 g.

### Example 22

3,125 g of any of the Areca catechu extracts in Examples 1 to 5 was added per ton of premixed feed having a cycle of concentration of 25 to obtain five biological feeds, and when the biological feed was mixed with an energy feed and a protein feed to prepare a complete feed, the proportion of the biological feed in the complete feed was 1/25, that is, the Areca catechu extract added per ton of complete feed was 125 g.

### Example 23

10,000 g of an Areca catechu ultrafine powder (80 meshes or more) was added per ton of premixed feed having a cycle of concentration of 25 to obtain a biological feed, and when the biological feed was mixed with an energy feed and a protein feed to prepare a complete feed, the proportion of the biological feed in the complete feed was 1/25, that is, the Areca catechu ultrafine powder (80 meshes or more) added per ton of complete feed was 400 g.

### Example 24

3,125 g of an Areca catechu ultrafine powder (80 meshes or more) was added per ton of premixed feed having a cycle of concentration of 25 to obtain a biological feed, and when the biological feed was mixed with an energy feed and a protein feed to prepare a complete feed, the proportion of the biological feed in the complete feed was 1/25, that is, the Areca catechu ultrafine powder (80 meshes or more) added per ton of complete feed was 125 g.

### Example 25

50,000 g of Areca catechu ultrafine powder (80 meshes or more) was added per ton of premixed feed having a cycle of concentration of 25 to obtain a biological feed, and when the biological feed was mixed with an energy feed and a protein feed to prepare a complete feed, the proportion of the biological feed in the complete feed was 1/25, that is, the Areca catechu ultrafine powder (80 meshes or more) added per ton of complete feed was 2,000 g.

### Example 26

25 g of any of the Areca catechu extracts in Examples 1 to 5 was added per ton of water, the resulting mixture was left to stand for 2 h for drinking ad libitum.

### Example 27

400 g of any of the Areca catechu extracts in Examples 1 to 5 was added per ton of water, the resulting mixture was left to stand for 2 h for drinking ad libitum.

### Example 28

According to a ratio of 125 g of Areca catechu ultrafine powder (80 meshes or more) per ton of water, the Areca catechu ultrafine powder was put into a filter bag, and the filter bag was soaked in water for 12 hours or more for drinking ad libitum.

### Example 29

According to a ratio of 2,000 g of Areca catechu ultrafine powder (80 meshes or more) per ton of water, the Areca catechu ultrafine powder was put into a filter bag, and the filter bag was soaked in water for 12 hours or more for drinking ad libitum.

### Example 30

25 g of any of the Areca catechu extracts in Examples 1 to 5 was added per ton of water, and the resulting mixture was sprayed in a pigsty environment.

### Example 31

400 g of any of the Areca catechu extracts in Examples 1 to 5 was added per ton of water, and the resulting mixture was sprayed in a pigsty environment.

It can be seen from the content described above that the use of the Areca catechu product as the sole active ingredient may block the feed-borne transmission and drinking water-borne transmission of ASFV. The method is simple and cost-effective, providing effective measures for the prevention and control of African swine fever.

Although the examples described above have provided a detailed description of the present disclosure, they are only a part of, rather than all of the embodiments of the present disclosure. All other embodiments that can be obtained according to the examples of the present disclosure without creative efforts shall fall within the protection scope of the present disclosure.

What is claimed is:

1. A method for preventing and controlling African swine fever, comprising administering a biological feed and/or a dinking preparation comprising an Areca catechu product as a sole active ingredient.

2. The method according to claim 1, wherein the Areca catechu product comprises Areca catechu extract and/or Areca catechu ultrafine powder.

3. The method according to claim 2, wherein polyphenol in the Areca catechu extract has a content of 2% to 30%; and
the Areca catechu ultrafine powder is an undersize of a sieve with meshes of ≥80.

4. The method according to claim 1, wherein a method for preparing the Areca catechu extract comprises the steps of:
mixing an Areca catechu raw material with an extraction solvent, performing extraction, and collecting a liquid extract of Areca catechu; and
subjecting the liquid extract of Areca catechu to drying to obtain the Areca catechu extract.

5. The method according to claim 4, wherein the Areca catechu product comprises Areca catechu extract and/or Areca catechu ultrafine powder.

6. The method according to claim 5, wherein polyphenol in the Areca catechu extract has a content of 2% to 30%; and
the Areca catechu ultrafine powder is an undersize of a sieve with meshes of ≥80.

7. The method according to claim 4, wherein
a mass-to-volume ratio of the Areca catechu raw material to the extraction solvent is 1 g:8 to 35 mL;
the drying is performed at a temperature of 65 to 75° C. for 72 to 96 h.

8. The method according to claim 7, wherein the extraction solvent is any one of water, methanol, and an aqueous hydrochloric acid solution having a mass concentration of 0.2 to 0.5%;
when the extraction solvent is water, the extraction comprises water bath extraction and the water bath extraction is performed at a temperature of 60 to 90° C. for 1 to 3 h;
when the extraction solvent is methanol, the extraction comprises ultrasonic extraction and the ultrasonic extraction is performed at a frequency of 60 to 100 Hz for 15 to 30 min; and
when the extraction solvent is an aqueous hydrochloric acid solution having a mass concentration of 0.2 to 0.5%, the extraction comprises: mixing the Areca catechu raw material with an aqueous hydrochloric acid solution having a mass concentration of 0.2 to 0.5%, soaking a resulting mixture for 1 h, loading the soaked mixture onto a percolation column, and performing percolation extraction with an aqueous hydrochloric acid solution having a mass concentration of 0.2 to 0.5%.

9. A biological feed for preventing and controlling African swine fever, wherein the biological feed consists of an Areca catechu product and basic feed; and
the basic feed comprises any one or more of a complete feed, a concentrated feed and a premixed feed.

10. The biological feed according to claim 9, wherein the Areca catechu product comprises Areca catechu extract and/or Areca catechu ultrafine powder;
polyphenol in the Areca catechu extract has a content of 2% to 30%;
when the Areca catechu product is Areca catechu extract, 25 g to 400 g of the Areca catechu extract is added per 1 ton of complete feed;
when the Areca catechu product is Areca catechu ultrafine powder, 125 g to 2,000 g of the Areca catechu ultrafine powder is added per 1 ton of complete feed; and
when the Areca catechu product is Areca catechu ultrafine powder and Areca catechu extract, 60 to 1,000 g of the Areca catechu ultrafine powder and 10 to 200 g of the Areca catechu extract are added per 1 ton of complete feed.

11. A dinking preparation for preventing and controlling African swine fever, wherein the dinking preparation consists of an Areca catechu product and water.

12. The dinking preparation according to claim 11, wherein the Areca catechu product comprises Areca catechu extract and/or Areca catechu ultrafine powder;
polyphenol in the Areca catechu extract has a content of 2% to 30%;
when the Areca catechu product is Areca catechu extract, 25 g to 400 g of the Areca catechu extract is added per 1 ton of water;
when the Areca catechu product is Areca catechu ultrafine powder, 125 g to 2,000 g of the Areca catechu ultrafine powder is added per 1 ton of water, and
when the Areca catechu product comprises Areca catechu ultrafine powder and Areca catechu extract, 60 to 1,000 g of the Areca catechu ultrafine powder and 10 to 200 g of the Areca catechu extract are added per 1 ton of water.
